(19) United States
(12) Patent Application Publication
(10) Pub. No.: US 2025/0001112 A1
(43) Pub. Date: Jan. 2, 2025

(54) TRACHEOSTOMY ANTI-DISCONNECT SYSTEM AND RELATED METHODS

(71) Applicant: REHABILITATION INSTITUTE OF CHICAGO D/B/A SHIRLEY RYAN ABILITYLAB, Chicago, IL (US)

(72) Inventors: Linda L. Morris, Chicago, IL (US); Christopher Conley, Chicago, IL (US)

(21) Appl. No.: 18/759,670

(22) Filed: Jun. 28, 2024

Related U.S. Application Data
(60) Provisional application No. 63/511,590, filed on Jun. 30, 2023.

(51) Int. Cl. A61M 16/04; A61M 39/10

(52) U.S. Cl. CPC A61M 16/0465; A61M 39/10; A61M 2039/1005

(57) ABSTRACT

An anti-disconnect system is provided for preventing unintended disconnection of a ventilator circuit from a tracheostomy tube of a patient in need of ventilation. A neck strap positioned around the neck of the patient, a ventilator holding element, and a closure element for releasably securing the ventilator holding element to the neck strap are provided.

### CROSS-REFERENCE TO RELATED APPLICATIONS

This is a Non-Provisional application that claims benefit to U.S. Provisional Application No. 63/511,590, filed Jun. 30, 2023, which is herein incorporated by reference in its entirety.

### FIELD

Aspects of the disclosure relate generally to methods and systems for preventing unintentional disconnection of a ventilator circuit from a tracheostomy tube of a ventilated patient.

### BACKGROUND

Ventilators are life-supporting medical devices that assist the breathing of individuals who are unable to breathe on their own. In some cases, a tracheostomy is performed to surgically create an opening through the front of the neck and into the trachea. A tracheostomy tube is then placed into the opening and connected to the ventilator. Some individuals continue to rely on the tracheostomy system and ventilator to breathe when discharged from the hospital.

While a ventilator-dependent patient is transferred from bed to wheelchair, attends therapy or participates in other activities of daily living, the ventilator circuit has the potential to disconnect from the tracheostomy tube, which could result in harm to the patient. When the ventilator tube is not secure, there is a risk of accidental decannulation (removal of the tube from the neck). Further, if disconnection occurs while the patient is sleeping or otherwise unattended, this can cause severe stress, respiratory distress or even death to the patient if unnoticed. Notably, the ventilator circuit is designed to pull out from the machine end of the tracheostomy tube with a pullout force less than 50 N in order to allow the nursing staff to suction the patient's trachea. This quick-disconnect feature is an important feature for daily maintenance; however, the downside is that it can inadvertently disconnect. Currently, there is no warning system or sensor that can alert the patient, caregivers or hospital staff that the ventilator has disconnected from the tracheostomy tube.

In practice, there is minimal guidance or information on methods to secure the tracheostomy tube. Currently, there are some known methods and commercial devices for preventing disconnection of the ventilator circuit from the tracheostomy tube, however each has limitations including difficulty of use by caregivers or nurses, discomfort to the patient, and ineffectiveness to prevent accidental disconnect. For example, the ventilator tube may be secured with cloth tape snugly placed around the neck of the patient (FIG. 1). One benefit of cloth tape is that it does not stretch; however, it can fold tightly and dig into the patient's skin, creating the potential for skin irritation. This solution also lacks reliability in that it can unintentionally come untied, is difficult for some caregivers to implement, and excess tape can interfere with patient movement, therapy or other caregiving.

Other commercially available anti-disconnect devices include the Ventilator Anti-Disconnect device by Marpac (FIG. 2), which comprises a fabric sling for holding the ventilator circuit and two hook and loop fastener tabs for connecting to the patient's tracheostomy neck belt. This device is not comfortable for patients in that it adds tension to the tracheostomy neck belt and the small hook and loop fastener tabs are often not strong enough to securely hold the ventilator circuit in place or prevent accidental disconnection.

The Stronghold Anti-disconnect device by Shiley directly connects the ventilator circuit to the tracheostomy tube (FIG. 3). The added bulk of this device and direct tension applied to the tracheostomy tube can be uncomfortable for the patient. Disconnection of the ventilator circuit is also known to occur with this device. The Pepper Medical Vent tie (FIG. 4), while simple, also adds uncomfortable pressure to the tracheostomy tube and the anti-disconnect straps are not sufficient to securely and reliably hold the ventilator circuit.

Thus, a need remains for a simple to use and lightweight device that reliably prevents disconnection of the ventilator circuit from the tracheostomy tube without adding extra weight to the circuit or adding pressure to the tracheostomy tube/neck flange.

It is with these observations in mind, among others, that various aspects of the present disclosure were conceived and developed.

### SUMMARY

The following presents a summary of various aspects described herein. This summary is not an extensive overview and is not intended to identify key or critical elements or to delineate the scope of the claims. The following summary merely presents some concepts in a simplified form as an introductory prelude to the more detailed description provided below.

The anti-disconnect system described herein presents a novel means for preventing unintentional disconnection of a tracheostomy assembly from a ventilator assembly. In one example, the ventilator anti-disconnect system includes a neck strap for being positioned around the neck of a patient, a ventilator holding element having a hole therein for accepting a portion of the ventilator assembly, and a closure element for releasably securing the ventilator holding element to the neck strap. The system may include at least a portion of the closure element at one or both ends of the neck strap and the ventilator holding element. In some examples of the system, the ventilator holding element may be affixed to an end of the neck strap. The ventilator holding element may be provided as a sling.

In a further embodiment, the neck strap of the ventilator anti-disconnect system includes at least a first segment affixed to a first end of the ventilator holding element and a second segment affixed to a second end of the ventilator holding element. At least a portion of the closure element may be connected to the second segment of the neck strap. The closure element may be provided as a tab having one of the hooks of a hook and loop fastener system and the loops of a hook and loop fastener system on a surface thereof.

In another example, a ventilator anti-disconnect system comprising a neck strap having a portion of a closure element at a first end thereof and a ventilator holding element is provided. A portion of a closure element may also be provided at a second end of the neck strap. Alternatively, the second end of the neck strap may be affixed to the ventilator closing element. In some examples, the closure element may be provided as at least one connector positioned on the neck strap and at least one tab positioned on the ventilator holding element. Further, the ventilator anti-disconnect system may also include a ventilator holding device having a sling portion with an opening at the center thereof configured to accept a portion of a ventilator circuit therethrough, and at least one tab configured to releasably engage the at least one connector. Various configurations are contemplated, including that the ventilator holding device has a tab at a first end of the sling portion for releasably engaging the at least one connector and a second end of the sling portion is permanently affixed to the strap. Alternatively, the ventilator holding device may include tabs at both ends of the sling portion for engaging connectors at both ends of the strap.

In operation, the strap is positioned around the neck of a patient and may be placed over the tracheostomy neck belt (“ties”) which secures the tracheostomy flange to the patient. The proximal side of the strap may include a hook and loop system (e.g., Velcro®) for releasably engaging the tracheostomy neck belt.

In some examples, the proximal side of the strap may be permanently affixed to the tracheostomy neck belt or may be provided as a single belt. In such examples, the neck belt may include at least one flange connector and at least one ventilator connector positioned proximal thereto.

A method for securing a ventilator circuit to a tracheostomy tube is also provided herein. In one aspect, the method includes the steps of: providing a strap configured to be positioned around the neck of a patient; providing at least one connector at an end of the strap; and providing a ventilator holding device having a sling portion with an opening at the center thereof configured to accept a portion of a ventilator circuit therethrough, and at least one tab configured to releasably engage the at least one connector.

These features, along with many others, are discussed in greater detail below.

### BRIEF DESCRIPTION OF THE DRAWINGS

The present disclosure is described by way of example and not limited in the accompanying figures in which like reference numerals indicate similar elements and in which:

FIG. 1 shows an example of the use of cloth tape to secure a ventilator circuit to a tracheostomy tube assembly;

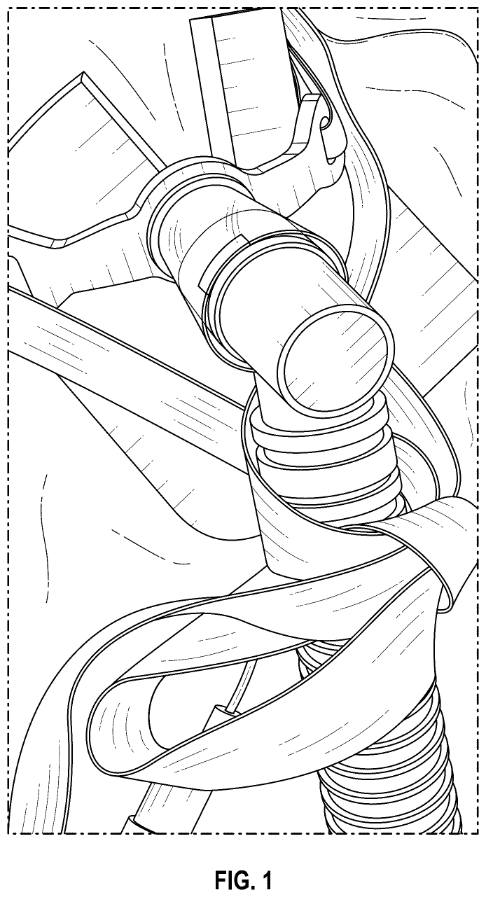

FIG. 2 show an example of the Ventilator Anti-Disconnect device by Marpac;

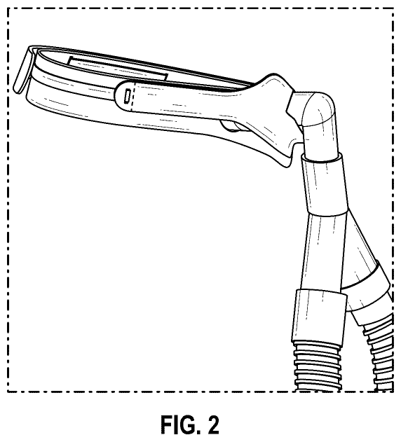

FIG. 3 shows an example of the Stronghold Anti-disconnect device by Shiley;

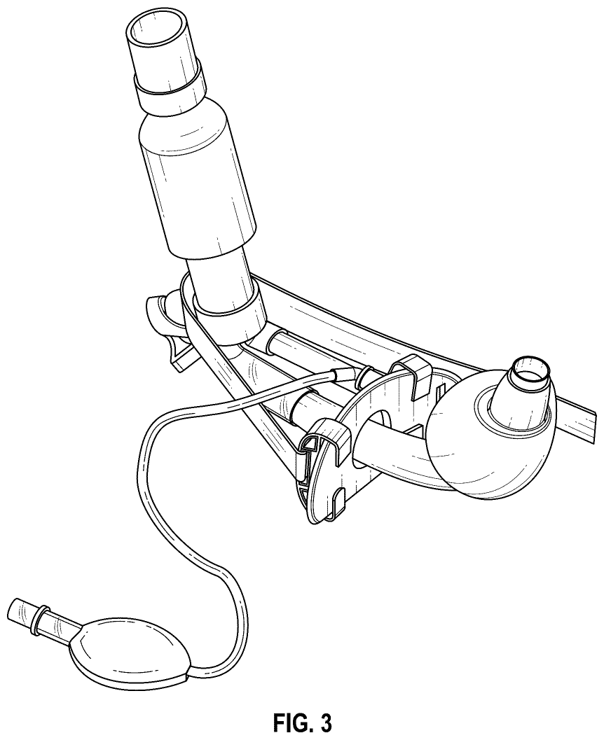

FIG. 4 shows an example of the Pepper Medical Vent tie;

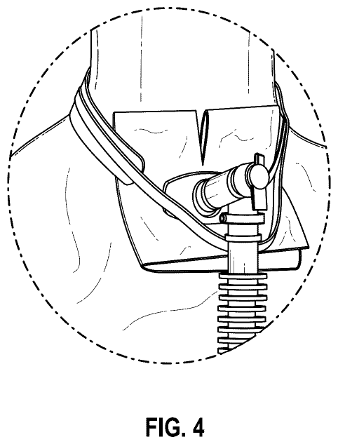

FIG. 5 shows an example of a tracheostomy tube assembly;

FIGS. 6A, 6B and 6C show an example of a ventilator anti-disconnect system, according to one or more aspects of the disclosure;

FIG. 7 shows an example of the anti-disconnect system of FIGS. 6A-6C, according to one or more aspects of the disclosure;

FIG. 8 shows an example of the anti-disconnect system of FIGS. 6A-6C, according to one or more aspects of the disclosure;

FIGS. 9A and 9B show an example of a ventilator anti-disconnect system in use on a mannequin, according to one or more aspects of the disclosure;

FIG. 10 shows an example of the ventilator anti-disconnect system of FIGS. 9A-9B, according to one or more aspects of the disclosure;

Corresponding reference characters indicate corresponding elements among the view of the drawings. The headings used in the figures do not limit the scope of the claims.

### DETAILED DESCRIPTION

Various aspects of an anti-disconnect system are described herein. In the following description, reference is made to the accompanying drawings, which form a part hereof, and which show by way of illustration various embodiments in which aspects of the disclosure can be practiced. It is to be understood that other embodiments can be utilized and structural and functional modifications can be made without departing from the scope of the present disclosure. Aspects of the disclosure are capable of other embodiments and of being practiced or being carried out in various ways. In addition, it is to be understood that the phraseology and terminology used herein are for the purpose of description and should not be regarded as limiting. Rather, the phrases and terms used herein are to be given their broadest interpretation and meaning.

Ventilated tracheostomy patients may be present in hospitals, rehabilitation and nursing facilities and home care settings. Patient movement and participation in regular daily activities may cause decannulation or disconnection of the ventilator circuit, if not secure. Caretakers and clinicians may not be able to constantly monitor connection of the tracheostomy tube to the ventilator and, currently, no notification or alarm system exists to alert caretakers of disconnection.

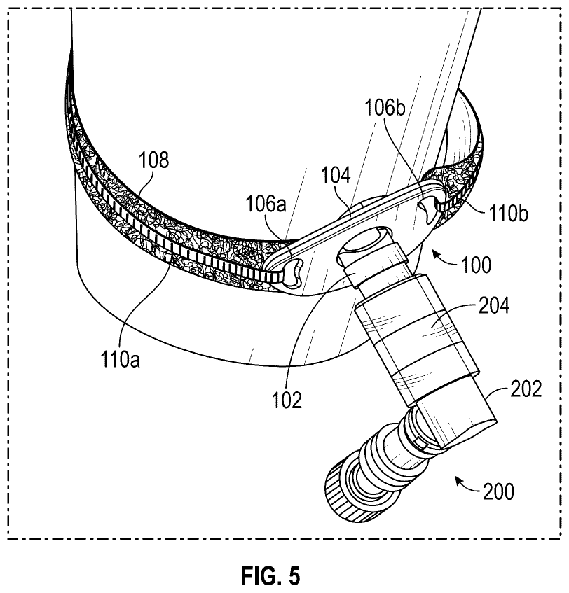

FIG. 5 illustrates relevant components of a tracheostomy assembly 100 and of a ventilator assembly 200. The tracheostomy assembly 100 includes a tracheostomy tube 102, which may include an outer cannula and an inner cannula, secured to a patient via a faceplate or flange 104. The flange 104 is intended to sit approximately flush with the neck of the patient and may include attachment points 106a, 106b at either end for a tie or collar 108 that is wrapped around the patient's neck, often called a tube tie or a tracheostomy collar. These terms may be used interchangeably herein. The tracheostomy collar 108, as shown in FIG. 5, may be covered in soft or felted fabric to be comfortable for the patient. If the tracheostomy collar 108 is in direct connection with the patient's body, it typically will need to be regularly replaced. For this reason, both ends of the collar 108 must be removable from the attachment points 106a, 106b of the flange 104 via a flange connector. In some examples, the flange connector is provided as tabs 110a, 110b at either end of the tracheostomy collar 108 that can be threaded through each of the attachment points 106a, 106b. One surface of the tabs 110a, 110b can be provided as or covered in the hooks of a hook and loop fastener, so as to releasably engage the soft or felted surface of the collar 108.

The ventilator assembly 200 can be connected to the tracheostomy assembly via a hub 202, using a connector 204, such as a conical connector. Disconnection of the ventilator assembly 200 from the tracheostomy assembly 100 is most likely to occur at the hub 202. In some examples, the connector 204 may be designed to disconnect with a pullout force of <50N to allow for ease of disconnection. While this quick disconnect feature enables clinical staff to perform daily respiratory care, for example, to suction a patient's trachea and breathing system through the inner cannula, it can also cause unintended disconnection.

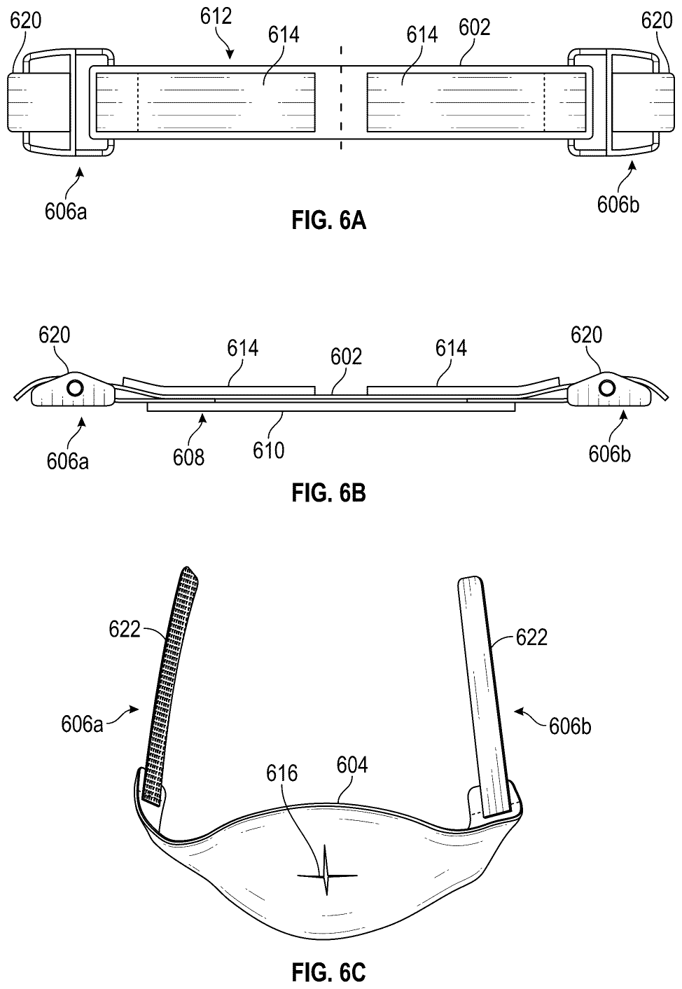

Embodiments of the anti-disconnect system described herein are designed to prevent unintentional disconnection of the ventilator assembly 200 from the tracheostomy assembly 100. FIGS. 6A-6C, FIG. 7 and FIG. 8 illustrate one embodiment of an anti-disconnect system 600 including a neck strap 602, a ventilator holding element 604, and at least one closure element 606.

In operation, the neck strap 602 of the anti-disconnect system is positioned around the posterior or back of the neck of a patient and provides several important benefits. As described above, a significant drawback of prior art devices and methods for securing a ventilator assembly to a tracheostomy tube lack sufficient stability to resist the counter force of the ventilator assembly. In practice, one or both of the small tabs with hook and loop fastener of the Marpac® device often fail and release from the fabric of the tracheostomy collar when under counter force from the ventilator assembly. Further, prior art devices or methods that secure the ventilator assembly directly to the forward-facing surface of the tracheostomy collar near the tracheostomy flange can disrupt or strain the tracheostomy tube itself, which can irritate the surrounding tissue and be uncomfortable for the patient.

By utilizing a dedicated neck strap 602, the anti-disconnect system 600 distributes the counter force of the ventilator assembly around the posterior neck of a patient rather than at or near the tracheostomy collar. Further, engaging a ventilator holding element 604 to a neck strap 602 via one or more closure elements 606 provides further stability and reliability to prevent unintentional disconnection of the ventilator circuit. The neck strap 602 may be made of one or more materials. For example, the strap 602 may be formed with an inner material that is strong and durable, and covered in a soft and breathable material that is comfortable and non-irritating. In another example, strap 602 may be formed of a single material having one or more of these characteristics.

In some embodiments, the neck strap 602 is intended to be positioned over the tracheostomy collar 108. The inner, or proximal, surface 608 of the strap 602 may be provided with one or more fasteners 610 for engaging the tracheostomy collar 108. The fastener 610 can prevent movement between the neck strap 602 and the tracheostomy collar 108 and can provide further stability to the system 600. The fastener 610 may, for example, be a continuous layer of, or may be one or more segments of, an element of a hook and loop fastener system or similar material. A layer or segments of the loop element of a hook and loop fastener system 614, or similar material, may be provided on the outer, or distal, surface 612 of the neck strap 602.

The ventilator holding element 604 may be any suitable device or combination of devices for engaging the ventilator assembly 200 and preventing disconnection from the tracheostomy assembly 100. In one embodiment, the ventilator holding element 604 includes a hole 616 therein for at least a portion of the ventilator assembly 200, such as the hub or 202 or connector 204 to pass through. As shown in FIG. 6C, the ventilator holding element 604 may be provided as a sling that wraps around the ventilator hub 202 or connector 204. The ventilator holding element 604 may be made of any suitable material. In some embodiments, the ventilator holding element 604 is made of a foam-backed fabric. Utilizing a tactile material such as foam may aid in engaging the ventilator circuit. Further, a flexible material may provide comfort for the patient, as it would allow for some give with movement of the patient.

The closure element 606 may be provided as any suitable device or material for securely and releasably connecting the ventilator holding element 604 to the neck strap 602 and that can be easily and rapidly operated and adjusted. For example, the closure element 606 may comprise or include hook and loop fastener, a snap, a buckle, or a strap fastener (e.g., a cam-locking lever fastener). The closure element 606 may be provided on one or both of the neck strap 602 and the ventilator holding element 604. In the embodiment of FIGS. 6A-6C, FIG. 7 and FIG. 8, each closure element 606a, 606b includes a connector 620 positioned on the neck strap 602 and a tab 622 positioned on the ventilator holding element 604. Each tab 622 may be inserted into and secured by connector 620. The inner surface of tab 622 may be lined with hook and loop fastener material for securing any portion of the tab that extends from the fastener to the corresponding hook and loop material 614 on the neck strap 602.

One of ordinary skill in the art will understand that other configurations or combinations of elements may be employed for the closure element 606. For example, closure element 606 may include a tab positioned on the neck strap and a connector positioned on the ventilator holding element. In another example, closure element 606 may include a tab with the hook surface of a hook and loop fastener positioned on the ventilator holding element that can directly engage the fabric of the neck strap.

The anti-disconnect system 600 may include one or more closure elements 606. In some embodiments, one end of the ventilator holding element 604 may be affixed to or integral with an end of the neck strap 602, such as by stitching, adhesive, or other means. The other end of the ventilator holding element 604 and/or neck strap 602 may be provided with a closure element 606. In the embodiment of FIGS. 6A-6C, FIG. 7 and FIG. 8, the anti-disconnect system 600 includes two closure elements 606a, 606b—one between either end of the ventilator holding element 604 and neck strap 602.

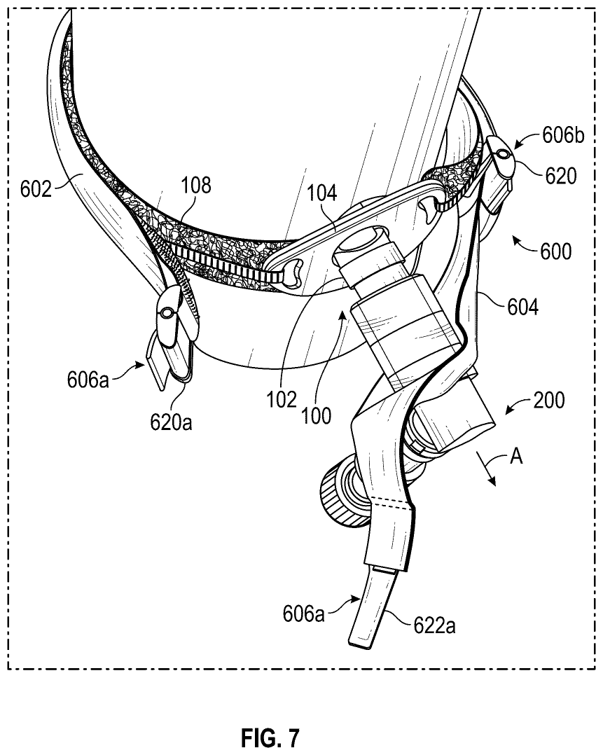

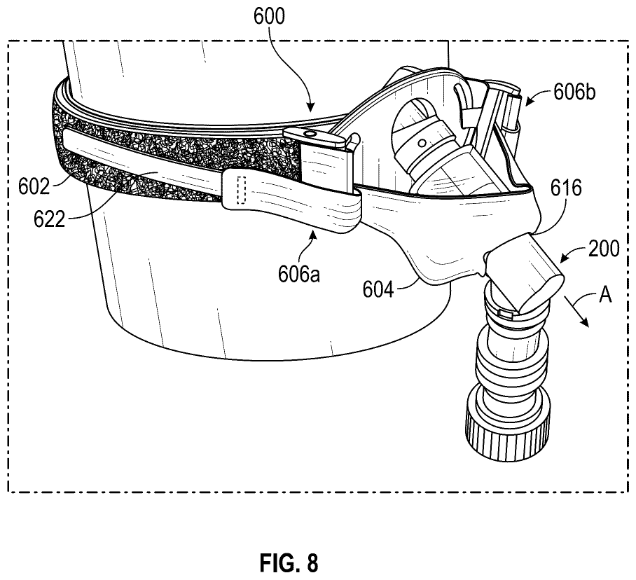

FIGS. 7 and 8 depict the anti-disconnect system 600 in operation in its open and closed positions, respectively. In an open position, at least one of the closure elements 606 is disengaged to allow for disconnection of the ventilator assembly 200 in the direction of arrow A. A caregiver or nurse may then perform daily maintenance activities on the patient's tracheostomy tube. In the illustrated embodiment, one tab 622a positioned on the ventilator holding element 604 is disengaged from its corresponding connector 620a on the neck strap 602. Both closure elements 606a, 606b may be disengaged if desired, for example, to replace or clean the ventilator holding element 604. In the fully closed position, shown in FIG. 8, the one or more closure elements 606 are engaged, thereby connecting the ventilator holding element 604 to the neck strap 602.

In some examples, the anti-disconnect system may be designed to not only secure the ventilator assembly to the tracheostomy assembly, but also to secure the tracheostomy assembly to the patient, thereby eliminating the need for a separate tracheostomy collar. In this example, the inner (patient facing) surface of the neck strap may be lined with a biocompatible and non-irritating material. Both ends of the neck strap may be provided with a flange connector for releasably engaging either side of the tracheostomy flange. In this embodiment, one or both ends of the neck strap and/or the ventilator holding element may also include a closure element for releasably connecting the ventilator holding element to the neck strap.

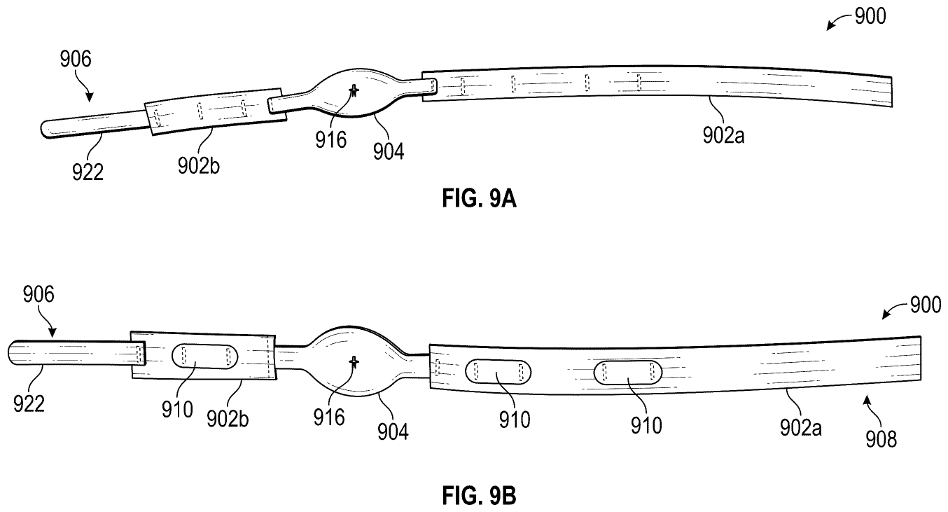

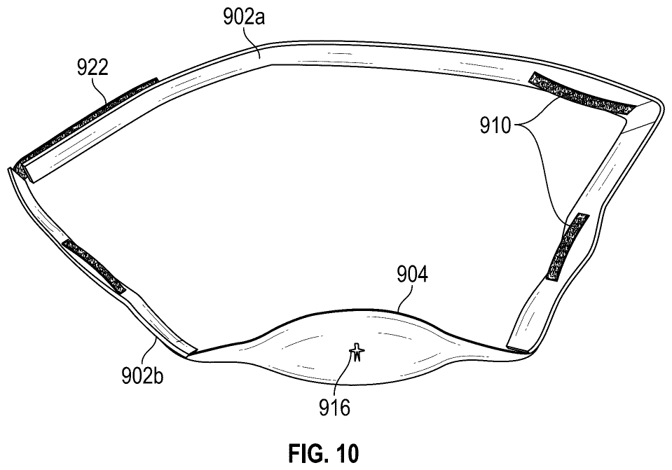

A further embodiment of the anti-disconnect system 900 is illustrated in FIGS. 9A-9B and FIG. 10, and includes a neck strap 902, a ventilator holding element 904 with a hole 916 therein for accepting at least a portion of a ventilator assembly, and a closure element 906. In this embodiment, the neck strap 902 includes a first segment 902a and a second segment 902b. The ventilator holding element 904 is affixed at a first end to the first segment 902a of the neck strap 902 and affixed at a second end to the second segment 902b of the neck strap 902. The closure element 904 comprises a tab 922 with the hook elements of hook and loop material on an inner surface for engaging the neck strap 902. In this example, the closure element 904 may also include a segment of the loop element of hook and loop material disposed on the neck strap 902 for engaging the tab 922. Alternatively, the neck strap 902 itself may be formed of or covered with a soft or felted material that can be directly engaged by the hooks on the tab 922. The ventilator holding element 904 may be provided as a sling, as shown in FIGS. 9A and 9B, similar to the sling described above with respect to system 600. One or more fasteners 910 may be disposed on a proximal surface 908 of the neck strap 902 for releasably engaging a tracheostomy collar.

In this example, tab 922 may be approximately the same length as the second segment 902b of the neck strap 902 to provide sufficient surface area to securely engage the first segment 902a. In this configuration, the closure element 906 is offset by some distance from the ventilator holding element 904, thereby connecting the first segment of the neck strap 902a to the second segment of the neck strap 902b and the ventilator holding element 904 at a position closer to the side of a patient's neck. This offset may allow the system 900 to further resist the counter force of the ventilator circuit, thereby better preventing unintentional disconnection.

One of ordinary skill in the art will recognize that the neck strap may be provided in three segments, wherein a longer segment is configured to be placed behind the back of the patient's neck, and two shorter segments of neck strap, with at least a portion of a closure element positioned thereon, are affixed at either end of the ventilator holding element. Other configurations are contemplated herein.

Examples of the anti-disconnect system described herein reliably prevent disconnection of the ventilator hub from the tracheostomy tube by tensioning the hub towards the machine (distal) end of the tracheostomy tube. As compared to prior art systems, the anti-disconnect system does not rely solely on small tabs with hook and loop fastener to hold the ventilator circuit against the tracheostomy neck belt. Rather, the anti-disconnect system includes at least one closure element for securely and releasably connecting the ventilator holding element to the neck strap.

Further, the anti-disconnect system as described herein is also comfortable for the patient. The ventilator holding element does not come into direct contact with the patient and therefore does not rub or otherwise irritate the patient's skin. Further, because the ventilator holding element is secured to the neck strap, which wraps around the back of the patient's neck, and not at or near the tracheostomy collar, the patient will not feel a sensation of tugging at the front of the neck belt. Hook-and-loop fastener, or equivalent material, on opposing surfaces of the neck belt and the tracheostomy collar can prevent relative movement between the two systems. The neck strap can be provided in various sizes to address variations in neck size. Further customization of the fit can be achieved by adjusting the one or more straps on the sling portion.

The anti-disconnect system is also convenient to apply and remove. The anti-disconnect system does not require daily change. However, because the tracheostomy collar touches the patient's skin, the belt must be changed with regularity, often daily. To change the tracheostomy collar, a caregiver disconnects at least one closure element on the anti-disconnect system and releases at least one end of the ventilator holding element from the neck belt. If present, fasteners on the neck belt can also be disengaged to release the neck belt from the tracheostomy collar, and the entire anti-disconnect system can be temporarily removed. The tracheostomy collar can then be removed and replaced.

Initial testing of the system produced positive results. Overall, patients reported that the system felt very secure and comfortable. Staff who applied the system found it to be easy to apply and secure. No unexpected or untoward events related to this system were reported. In one pilot trial, the patient was placed in a variety of positions: sitting up, lying supine, side-lying left and right, head turned up and down, right and left. The patient reported that the ventilator tube felt “very secure”, “doesn't move”, “not too heavy”, “not abrasive”, “stays in place”, and was not at all uncomfortable.

Although the present invention has been described in certain specific aspects, many additional modifications and variations would be apparent to those skilled in the art. It is therefore to be understood that the present invention can be practiced otherwise than specifically described without departing from the scope and spirit of the present invention. Thus, embodiments of the present invention should be considered in all respects as illustrative and not restrictive.

What is claimed is:

1. A ventilator anti-disconnect system comprising:
a neck strap;
a ventilator holding element having an opening therein configured to accept a portion of a ventilator circuit therethrough; and
a closure element configured to releasably connect the ventilator holding element to the neck strap.

2. The ventilator anti-disconnect system of claim 1 wherein a proximal side of the neck strap includes at least one fastener for engaging a tracheostomy collar positioned on a neck of a patient.

3. The ventilator anti-disconnect system of claim 2 wherein the at least one fastener comprises at least one hook of a hook and loop fastener system and at least one loop of the hook and loop fastener system.

4. The ventilator anti-disconnect system of claim 1, further comprising two closure elements.

5. The ventilator anti-disconnect system of claim 1, wherein an end of the ventilator holding element is affixed to an end of the neck strap.

6. The ventilator anti-disconnect system of claim 1 wherein:
the neck strap comprises a first segment affixed to a first end of the ventilator holding element and a second segment affixed to a second end of the ventilator holding element; and
at least a portion of the closure element is connected to the second segment of the neck strap.

7. The ventilator anti-disconnect system of claim 6 wherein the closure element comprises a tab having at least one hook of a hook and loop fastener system and at least one loop of the hook and loop fastener system on a surface thereof.

8. The ventilator anti-disconnect system of claim 1 wherein the closure element comprises:
at least one tab positioned on the ventilator holding element; and
at least one connector positioned on the neck strap and configured to releasably engage the at least one tab.

9. The ventilator anti-disconnect system of claim 8, wherein the closure element further comprises:
a first tab and a second tab positioned on the ventilator holding element;
a first connector positioned at a first end of the neck strap and configured to releasably engage the first tab; and
a second connector positioned at a second end of the neck strap and configured to releasably engage the second tab.

10. The ventilator anti-disconnect system of claim 8 further comprising:
a first flange connector positioned at a first end of the neck strap; and
a second flange connector positioned at a second end of the neck strap;
wherein each of the first flange connector and the second flange connector are configured to engage a tracheostomy flange.

11. The ventilator anti-disconnect system of claim 1, wherein the ventilator holding element is a sling.

12. A ventilator anti-disconnect system comprising:
a neck strap having a first segment and a second segment, said neck strap configured to be positioned around a neck of a patient;
a ventilator holding element having an opening therein configured to accept a portion of a ventilator circuit therethrough, a first end of the ventilator holding element connected to the first segment of the neck strap and a second end of the ventilator holding element connected to the second segment of the neck strap; and
a closure element connected to the second segment of the neck strap configured to releasably engage the first segment of the neck strap.

13. The ventilator anti-disconnect system of claim 12, further comprising:
at least one fastener positioned on a proximal side of the neck strap configured to releasably engage a tracheostomy collar positioned on the neck of the patient.

14. The ventilator anti-disconnect system of claim 13, further comprising:
at least one fastener positioned on a proximal side of the first segment of the neck strap; and
at least one fastener positioned on a proximal side of the second segment of the neck strap.

15. The ventilator anti-disconnect system of claim 12, wherein the closure element comprises a tab including at least one hook of a hook and loop fastener system and at least one loop of the hook and loop fastener system.

16. A method of securing a ventilator circuit to a tracheostomy tube, comprising steps of:
positioning a ventilator anti-disconnect system around a neck of a patient, wherein the ventilator anti-disconnect system comprises:
a neck strap,
a ventilator holding element having an opening therein for accepting a portion of a ventilator circuit therethrough, and
at least one closure element associated with the ventilator holding element configured to releasably engage the neck strap;
inserting a portion of a ventilator circuit through the opening in the ventilator holding element;
connecting the ventilator circuit to a tracheostomy tube; and
engaging the ventilator holding element to neck strap with the at least one closure element.

17. The method of securing a ventilator circuit to a tracheostomy tube of claim 16, wherein a proximal side of the neck strap includes at least one fastener for engaging a tracheostomy collar positioned on the neck of the patient.

18. The method of securing a ventilator circuit to a tracheostomy tube of claim 16, wherein an end of the ventilator holding element is affixed to an end of the neck strap.

19. The method of securing a ventilator circuit to a tracheostomy tube of claim 18, wherein:
the neck strap comprises a first segment affixed to a first end of the ventilator holding element and a second segment affixed to a second end of the ventilator holding element; and
at least a portion of the at least one closure element is connected to the second segment of the neck strap.

20. The method of securing a ventilator circuit to a tracheostomy tube of claim 19, wherein the at least one closure element comprises a tab having at least one hook of a hook and loop fastener system and at least one loop of the hook and loop fastener system on a surface thereof.
